(19) United States
(12) Patent Application Publication
(10) Pub. No.: US 2018/0235860 A1
(43) Pub. Date: Aug. 23, 2018

(54) USE OF DIETHANOLAMMONIUM DERIVATIVES AS SKIN MOISTURIZER

(71) Applicant: L'OREAL, Paris (FR)

(72) Inventors: Maria DALKO, Versailles (FR); Julien HITCE, Aulnay-sous-Bois (FR)

(21) Appl. No.: 15/752,034

(22) PCT Filed: Aug. 16, 2016
(86) PCT No.: PCT/EP2016/069387

§ 371 Date: Feb. 12, 2018

(30) Foreign Application Priority Data
Aug. 17, 2015 (FR) 1557771

(51) Int. Cl. A61K 8/41; A61Q 19/00

(52) U.S. Cl. CPC A61K 8/416; A61K 2800/40; A61Q 19/007

(57) ABSTRACT

The present invention relates to the cosmetic use as moisturizer for keratin materials, preferably the skin, of one or more diethanolammonium derivatives corresponding to formula (I) below, and also the optical isomers and/or geometrical isomers thereof: (I)

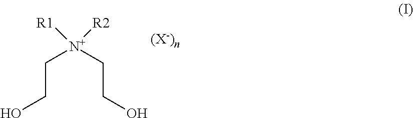

The present invention relates to the use of diethanolammonium derivatives in the field of caring for keratin materials such as the skin, and especially as a moisturizer for keratin materials, preferably the skin.

The skin, which is a protective and exchange barrier with the environment, is both strong and fragile, it may lose its suppleness and its capacity to retain water decreases, then causing skin dryness.

It is known that the stratum corneum or corneal layer, which is the outermost region of the epidermis, is most particularly involved in moisturizing the skin. Located at the interface with the external environment, its function is especially to delay excessive water loss arising from the deeper layers of the epidermis. The stratum corneum also protects against mechanical attack. It also constitutes the first line of defence against UV radiation.

With a thickness of 10 μm, it is composed of vertically stacked corneocytes surrounded by a matrix of lipid-enriched membranes. Thus, it is a two-compartment system that may be compared to a wall of bricks, composed of anuclear cells (the “bricks”) and of intercellular lamellar membranes (the “cement”). By virtue of this compact stratified structure, the stratum corneum performs its barrier function by opposing transcutaneous water loss. It thus efficiently contributes towards the moisturization of the skin via its capacity to take up and retain water, which is mainly located in the intercellular spaces.

For obvious reasons, it is important to ensure a sufficient level of skin moisturization in order to preserve its suppleness, softness, tonicity and/or appearance. In general, a decrease in this moisturization may be prevented or treated by acting on the stratum corneum via moisturizers, for instance glycerol, which are the reference active agents in this field.

However, drawbacks are observed with active agents of these types.

For example, glycerol has the drawback of making formulations tacky, especially when they are used in high concentration. There is thus a need to find other active agents in the field of skin moisturization that are free of the abovementioned drawbacks.

The inventors have discovered that diethanolammonium derivatives of general formula (I) as described below are good moisturizers, and especially have a beneficial effect in terms of elasticity of the stratum corneum and/or improve the barrier function. The diethanolammonium derivatives of general formula (I), which are the subject of the present invention, make it possible especially to provide, while at the same time having a moisturizing effect, compositions that have good stability and/or that remain pleasant for the consumer, i.e. being sparingly tacky, having a pleasant feel, and/or having no discomfort sensations such as tautness.

Thus, one subject of the present invention is the cosmetic use of one or more diethanolammonium derivatives corresponding to formula (I) below, and also the optical isomers and/or geometrical isomers thereof:

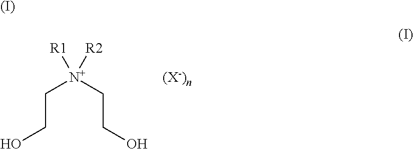

in which

R1 and R2 independently denote:

(i) a linear or branched C1-C12 alkyl radical which is saturated or unsaturated with one or more ethylenic double bond(s); said radical being optionally substituted with a group —COR; or

(ii) an aralkyl radical CmH2mAr such that m denotes an integer ranging from 1 to 6 and Ar denotes an aryl radical optionally substituted with a group —COR;

R denotes a group —OR′ or —NR′R″ where R′ and R″ independently denote hydrogen or a linear or branched C1-C6 alkyl radical which is saturated or unsaturated with an ethylenic double bond;

n is equal to 0 or 1;

the electrical neutrality of said compounds being provided by an organic or inorganic external anion X— (n=1) or internally (n=0) when a single COOH radical is present and forms a CO2- radical;

it being understood that when R1 denotes a methyl radical and R2 denotes a methyl radical substituted with a —COOH group, then n=1;

as a moisturizer for keratin materials, preferably the skin, and especially for treating dry skin.

The compound(s) of formula (I), particularly the compound(s) of formulae (II) and/or (III) that will be described herein below, according to the invention especially promote the persistence of a moisturizing treatment. The compound(s) of formula (I), in particular the compound(s) of formulae (II) or (III), according to the invention more especially have satisfactory moisturizing activity after 6 hours, or even after 24 hours of a single application thereof to keratin materials, such as the skin.

These moisturizing properties of the compounds of formula (I) according to the invention, particularly of the compounds of formulae (II) and/or (III), are especially afforded to the skin in a long-lasting manner after repeated application, i.e. they persist for several days and/or one or more weeks after application, even if the keratin materials, such as the skin, are no longer in contact with the compounds according to the invention or the compositions comprising them according to the invention.

The compound(s) of formula (I) according to the invention, particularly the compound(s) of formulae (II) and/or (III), or the compositions comprising them, may be used once or repeatedly, such as one to two times per day, preferably once a day, preferably over a period of at least one week, and more particularly of at least four weeks.

The compound(s) of formula (I), in particular the compound(s) of formula (II) and/or (III), as defined, may be used, alone or as mixtures, as moisturizer, in a composition comprising a physiologically acceptable medium, in particular a cosmetically acceptable medium.

In other words, the invention relates to the use of one or more compounds of formula (I), (II) and/or (III), as defined previously, as moisturizer, alone or as a mixture, especially in a composition comprising a physiologically acceptable medium, in particular a cosmetically acceptable medium.

Similarly, the invention relates to the use of said composition for moisturizing keratin materials, preferably the skin, and more particularly for treating dry skin.

The invention also relates to a composition comprising, in a physiologically acceptable medium, one or more compounds of formula (I) as defined previously and one or more additional moisturizing active agents, these additional moisturizing active agents being other than the compounds of formula (I).

Moreover, the invention also relates to novel specific compounds of formula (II) and/or (III) as defined herein below and also to the compositions containing them, especially compositions in a physiologically acceptable medium.

Another subject of the present invention relates to a cosmetic process for moisturizing keratin materials, in particular the skin, which consists in applying to a human keratin material, preferably to the skin, one of the compositions as defined previously. Preferably, the keratin materials, such as the skin, are human keratin materials.

Other characteristics, subjects and advantages of the invention will emerge more clearly on reading the description and the examples that follow.

In the text herein below, and unless otherwise indicated, the limits of a range of values are included within that range.

The expression “at least one” is equivalent to the expression “one or more”.

For the purposes of the present invention, the term “physiologically acceptable medium” is intended to denote a medium that has no unpleasant odour or appearance, and that is entirely compatible with the topical administration route. In the present case in which the composition is intended for topical administration, i.e. by application to the surface of the keratin material under consideration, such a medium is considered in particular to be physiologically acceptable when it does not cause discomfort, such as stinging or tautness that is unacceptable to the user.

In the present invention, the term “keratin material” means a keratin material, preferably a human keratin material, and in particular the skin (preferably human skin), and even more particularly bodily and/or facial skin.

The cutaneous region may be chosen in particular from:
the hands,
the face, in particular the forehead, the cheeks or the contour of an eye (periocular), and in particular the crow's feet, the region below the eye (bag), or the eyelids,
the neck,
the feet,
the legs,
the arms and forearms.

### I. Diethanolammonium Derivatives

The diethanolammonium derivatives in accordance with the present invention correspond to formula (I) below, and also the optical isomers and/or geometrical isomers thereof:

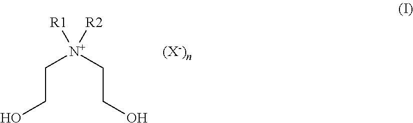

in which:

R1 and R2 independently denote:

(i) a linear or branched C1-C12 alkyl radical which is saturated or unsaturated with one or more ethylenic double bond(s); said radical being optionally substituted with a group —COR; or

(ii) an aralkyl radical CmH2mAr such that m denotes an integer ranging from 1 to 6 and Ar denotes an aryl radical such as phenyl, naphthyl, furyl or indolyl, optionally substituted with a group —COR;

R denotes a group —OR′ or —NR′R″ where R′ and R″ independently denote hydrogen or a linear or branched C1-C6 alkyl radical which is saturated or unsaturated with one or more ethylenic double bonds;

n is equal to 0 or 1;

the electrical neutrality of said compounds being provided by an organic or inorganic external anion X— (n=1) or in the form of an internal salt (n=0) when a single COOH radical is present and forms a CO2- radical (presence of a single COR radical with R═OR′ and R′═H, namely of a single CO2H radical in CO2- form where the anion X— is non existent);

it being understood that when R1 denotes a methyl radical and R2 denotes a methyl radical substituted with a —COOH group, then n=1.

Advantageously, the anion X⁻ is chosen such that the compound of formula (I) is physiologically acceptable.

In a particular embodiment, the diethanolammonium derivatives according to the invention correspond to the formula (I) as mentioned above in which R1 and R2 independently denote:

(i) a linear or branched C1-C12 alkyl radical which is saturated or unsaturated with one or more ethylenic double bond(s); said radical being optionally substituted with a group —COR; or

(ii) an aralkyl radical CmH2mAr such that m denotes an integer ranging from 1 to 6 and Ar denotes an aryl radical chosen among phenyl, naphthyl, or furyl, optionally substituted with a group —COR; and more particularly the aryl radical group is phenyl optionally substituted with a group —COR.

For the purposes of the invention, the term “physiologically acceptable compound” is intended to denote any compound of formula (I) or (II) or (III) that is suitable for the topical administration of a composition in which it is present.

A physiologically acceptable compound of formula (I) or (II) or (III) is preferentially a compound of formula (I) or (II) or (III) that has no unpleasant odour or appearance and that is entirely compatible with the topical administration route. In the present case in which the composition is intended for topical administration, i.e. by application to the surface of the keratin material under consideration, such a compound is considered in particular to be physiologically acceptable when it does not cause any discomfort, such as stinging or tautness that is unacceptable to the user.

X⁻ denotes an organic or mineral anion.

The term “organic anion X⁻” means the anionic forms of organic compounds bearing one or more acid functions (in particular carboxylic acid or sulfonic acid) such as the anionic forms of amino acids (carboxylates such as aspartate), or methanesulfonate, para-toluenesulfonate, camphorsulfonate, tartrate, citrate, acetate, levulinate or methosulfate.

In an embodiment when “organic anion X⁻”—denotes an anionic form of organic compounds bearing one or more carboxylic acid functions, it chosen among compounds of formula A or B or C as defined below:

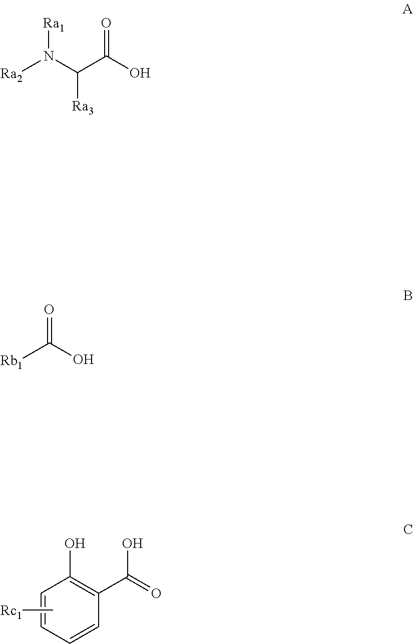

With

Ra1 means a hydrogen atom or an a linear or branched alkyl radical C1-C18

Ra2 means a hydrogen atom or an a linear or branched alkyl radical C1-C18 or an a linear or branched alkylcarbonyl radical C1-C18

Ra1 and Ra2 can together form a five to eight membered ring with the N atom bearing them.

Ra2 and Ra3 can together form a five membered ring of formula A1 or A2 with the N atom bearing them.

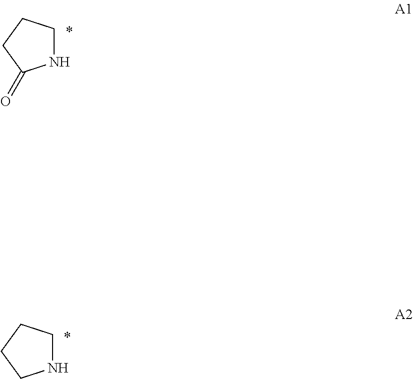 [SMILES: O=C1CCCN1 ; C1CCNC1]

Ra3 is chosen among the radical described below

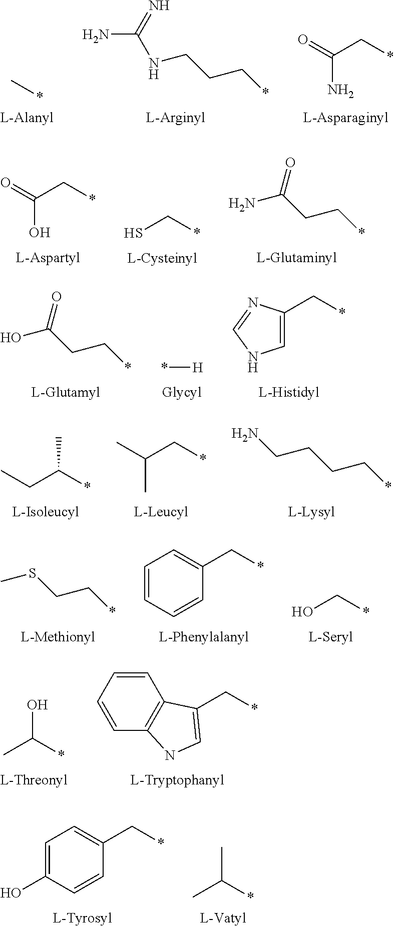

Preferentially

Ra1 means a hydrogen atom

Ra2 means a hydrogen atom or an a linear or branched alkyl radical C1-C8 or an a linear or branched alkylcarbonyl radical C1-C8

Ra2 and Ra3 can together forma five membered ring of formula A1 or A2 with the N atom bearing them.

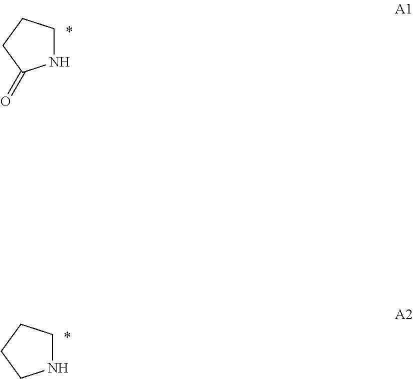 [SMILES: O=C1CCCN1 ; C1CCNC1]

Ra3 is chosen among the radical described below

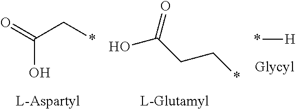

R1b means a linear or branched alkyl radical C1-C6 preferably C1-C4 substituted by at least one OH radical preferably one 011 radical or an saturated heterocycle of 5 to 8 membered ring containing one atom of N, S, O and substituted by at least one OH radical preferably one OH radical.

Preferentially,

R1b means a linear or branched alkyl radical C1-C4 substituted by one OH radical or a pyrrolidine containing substituted by one OH radical.

More preferably,

R1b is chosen among —CH2OH, —CH(OH)Me,

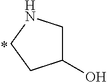

R1c means a linear or branched alkyl radical C1-C18 or a linear or branched alkyl carbonyl C1-18

Preferentially, R1c fits with compounds described in FR3012960 (as the anionic part) and FR2581542.

More preferably, R1c is corresponding to the 2 compounds described below

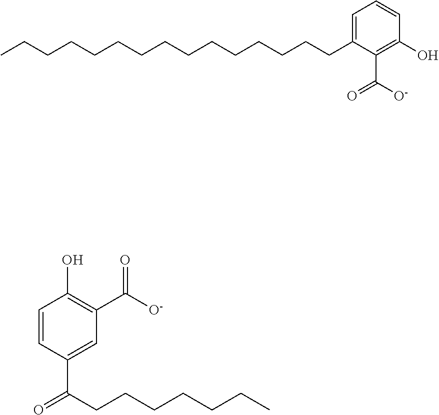 [SMILES: CCCCCCCCCCCCCCCc1cccc(O)c1C(=O)[O-] ; CCCCCCCC(=O)c1ccc(O)c(C(=O)[O-])c1]

When X— denotes an anionic form of organic compounds bearing one or more carboxylic acid functions it could also be chosen among anionic forms of compounds described in either FR2835525 or FR2810033.

In this embodiment, preferred X— are described below:

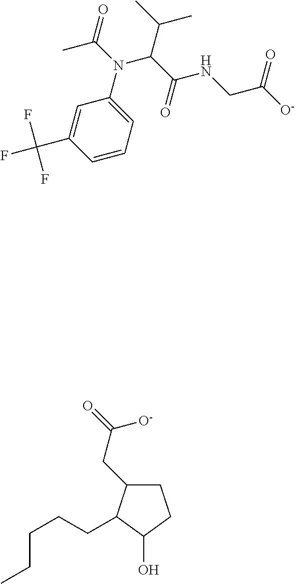 [SMILES: CC(=O)N(c1cccc(C(F)(F)F)c1)C(C(=O)NCC(=O)[O-])C(C)C ; CCCCCC1C(O)CCC1CC(=O)[O-]]

Preferentially when X— denotes an anionic form of organic compounds bearing one or more carboxylic acid functions, they are chosen among compounds of formula B.

In another embodiment, the term “mineral anion X—” means, for example, halides, in particular chloride, bromide or iodide, sulfates, phosphates, hydrogen phosphates, dihydrogen phosphates, carbonate or hydrogen carbonate.

Preferably, X⁻ is an anion chosen from halides, in particular chloride, bromide or iodide, sulfates, phosphates, hydrogen phosphates, dihydrogen phosphates, carbonate, hydrogen carbonate, methanesulfonate, para-toluenesulfonate, camphorsulfonate, tartrate, citrate, acetate, levulinate or aspartate.

X⁻ denotes more preferentially an inorganic anion and even more preferentially a halide, in particular bromide, iodide or chloride, levulinate or else a methosulfate anion.

Most particularly, X⁻ is an anion chosen from halides, in particular chloride, bromide or iodide.

As examples of compounds of formula (I), mention may be made of the following compounds:

| Compounds | Structure |
|---|---|
| a | 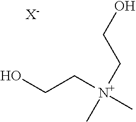 |
| b | 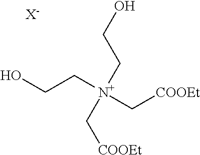 [SMILES: CCOC(=O)C[N+](CCO)(CCO)CC(=O)OCC ; "X-"] |
| c | 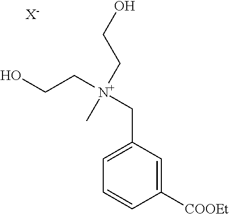 [SMILES: CCOC(=O)c1cccc(C[N+](C)(CCO)CCO)c1 ; "X-"] |
| d | 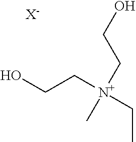 |
| e | 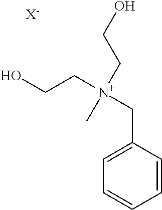 |
| f | 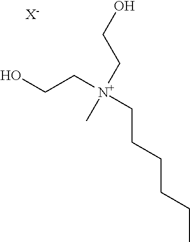 |
| g | 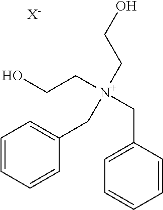 [SMILES: OCC[N+](CCO)(Cc1ccccc1)Cc1ccccc1 ; "X-"] |

where X⁻ denotes an organic or inorganic anion as previously indicated.

In particular, the compounds of formula (I) are chosen from the following compounds (1) to (24) and most particularly compounds (2), (3), (13) and (14), and even more preferentially compound (2):

| Compound | Structure |
|---|---|
| 1 | 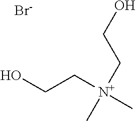 |
| 2 | 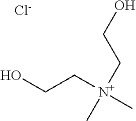 |
| 3 | 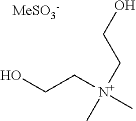 |
| 4 | 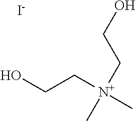 |
| 5 | 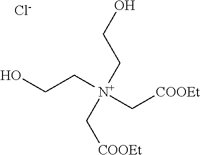 [SMILES: CCOC(=O)C[N+](CCO)(CCO)CC(=O)OCC ; [Cl-]] |
| 6 | 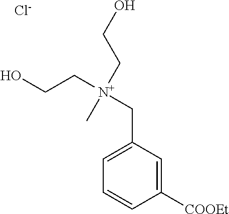 [SMILES: CCOC(=O)c1cccc(C[N+](C)(CCO)CCO)c1 ; [Cl-]] |
| 7 | 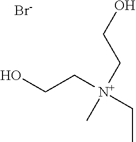 |
| 8 | 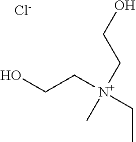 |
| 9 | 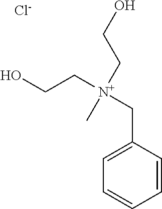 |
| 10 | 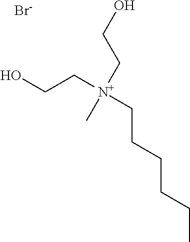 |
| 11 | 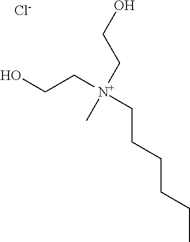 |
| 12 | 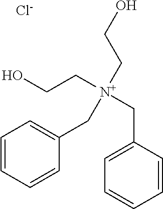 [SMILES: OCC[N+](CCO)(Cc1ccccc1)Cc1ccccc1 ; [Cl-]] |
| 13 | 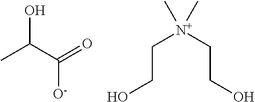 |
| 14 | 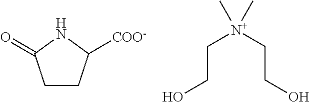 |
| 15 | 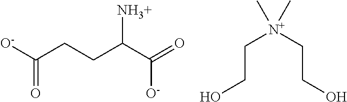 |
| 16 | 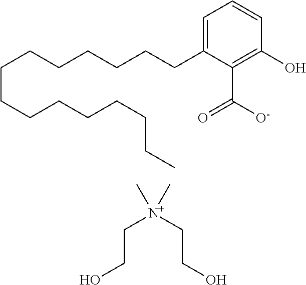 |
| 17 | 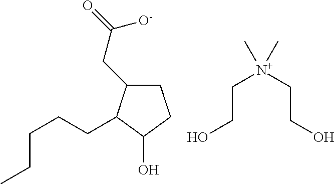 |
| 18 | 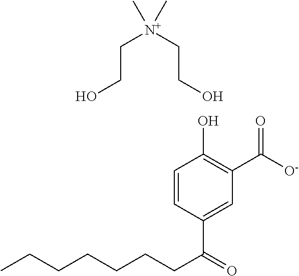 |
| 19 | 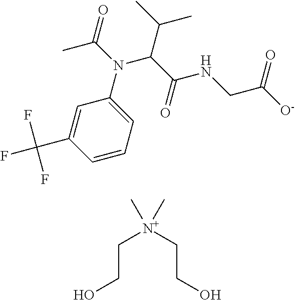 |
| 20 | 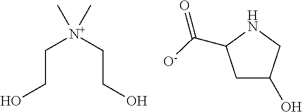 |
| 21 | 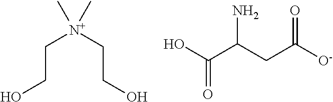 |
| 22 | 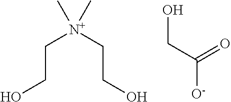 |
| 23 | 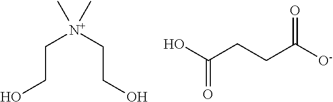 |
| 24 | 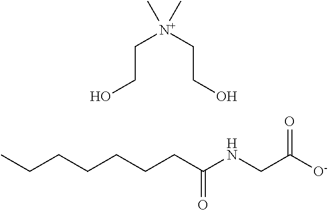 |

According to a preferred variant of the invention, the compounds of formula (I) are such that R1 and R2 independently denote a linear or branched C₁-C₈ alkyl radical which is saturated or unsaturated with one or more ethylenic double bond(s), and which is optionally substituted with a group —COR where R has the same meaning as indicated previously.

According to this variant, the compounds that will even more particularly be chosen are those for which R1 and R2 independently denote a linear or branched, saturated C₁-C₈ alkyl radical which is optionally substituted with a group —COR where R denotes a radical chosen from —OR′ and —NR′R″, with R′ and R″ independently denoting hydrogen or a linear or branched, saturated C₁-C₆ alkyl radical.

According to this variant, the compounds of formula (I) particularly preferably denote compounds (a), (b), (d) and (f) described previously and even more preferentially compounds (1), (2), (3), (4), (5), (7), (8), (10) and (11) described previously.

### II. Production of the Diethanolammonium Derivatives

The diethanolammonium derivatives in accordance with the invention may be obtained by quaternization of the corresponding amine in the presence of an excess of alkyl halide. Moreover, certain ethanolammonium derivatives that form the subject of the present invention are commercially available. For example, compound (1) is available from the supplier Acros Organics (France) under the reference 402530250.

In the case of the symmetrical compounds of formula (I), i.e. in which the radicals R1 and R2 are identical, N,N-diethanolamine, dissolved in an organic solvent (such as methanol, ethanol, tetrahydrofuran, N,N-dimethylformamide, etc.) is treated with an excess of alkyl halide R1-X in which R1 and X have the same meanings indicated previously in formula (I), at a temperature ranging from 0 to 150° C. for 1 to 24 hours according to the reaction scheme below:

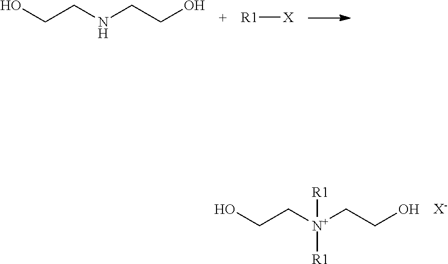

In the case of the asymmetrical compounds of formula (I), i.e. in which the radicals R1 and

R2 are different, N,N-diethanolamine, dissolved in an organic solvent (such as methanol, ethanol, tetrahydrofuran, N,N-dimethylformamide, etc.) is treated with one equivalent of alkyl halide R1-X in which R1 and X have the same meanings indicated previously in formula (I), at a temperature ranging from 0 to 150° C. for 1 to 24 hours.

The product resulting from this first alkylation is then dissolved in an organic solvent (such as methanol, ethanol, tetrahydrofuran, N,N-dimethylformamide, etc.) and treated with an excess of alkyl halide R2-X in which R2 and X have the same meanings indicated previously in formula (I), at a temperature ranging from 0 to 150° C. for 1 to 24 hours.

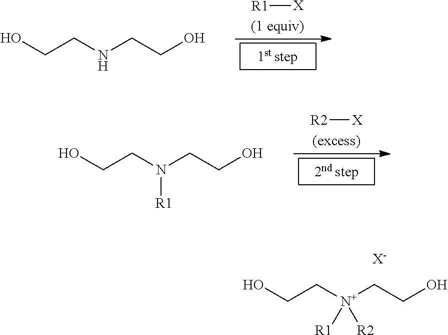

In the case where R1 is methyl, the intermediate N-methyl-N,N-diethanolamine is commercially available (for example Acros Organics France) and it can be used directly in the second step.

A subject of the invention is also novel compounds of formula (II) or (III) as defined below.

Moreover, a subject of the invention is also a composition comprising, especially in a physiologically acceptable medium, one or more compounds of formula (II) and/or one or more compounds of formula (III) as defined herein below.

Among the compounds of formula (I) in accordance with the invention, some have been described in U.S. Pat. No. 4,892,728 and FR 2 065 371 in compositions comprising a physiologically acceptable medium. Other compounds of formula (I) have never been used in a physiologically acceptable medium. This is the case for compounds (d), (f) and (g) as defined previously (irrespective of the nature of X⁻) and for compounds (e) as defined previously, for which X is other than OH⁻.

More particularly, this is the case for compounds (1), (2), (3), (4), (7), (8), (9), (10) and (11) as defined previously.

One subject of the invention is thus a composition comprising, in a physiologically acceptable medium, in particular a cosmetically acceptable medium, one or more compounds chosen from:
compounds (d), (f) and (g) as defined previously and
compounds (e) as defined previously for which X⁻ is other than OH⁻.

Another subject of the invention is a composition comprising, in a physiologically acceptable medium, in particular a cosmetically acceptable medium, one or more compounds chosen from compounds (1), (2), (3), (4), (7), (8), (9), (10), (11), (13) and (14) as defined previously.

### III. Novel Diethanolammonium Derivatives of Formulae (II) and (III)

Among the compounds of formula (I) in accordance with the invention, some are known, and others are novel and constitute another subject of the invention.

Among the novel compounds of formula (I), mention may be made of the compounds of formula (II) below, and also the optical isomers and/or geometrical isomers thereof:

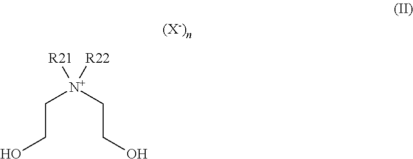

in which:

R21 denotes a linear or branched C₁-C₁₂ alkyl radical which is saturated or unsaturated with an ethylenic double bond;

R22 denotes a CₘH₂ₘAr aralkyl radical, where m is an integer ranging from 1 to 6 and Ar is an aryl radical substituted with a COR group, in particular a phenyl radical substituted with a COR group;

R has the same meaning previously indicated in formula (I);

n is equal to 0 or 1;

the electrical neutrality of said compounds being ensured by an external anion X⁻ (n=1) having the same meaning indicated previously in formula (I) or in the form of an internal salt (n=0) when a single COOH radical is present and forms a CO₂⁻ radical;

it being understood that when R1 denotes a methyl radical and R2 denotes a methyl radical substituted with a —COOH group, then n=1;

said compounds being other than the compounds having the following formulas:

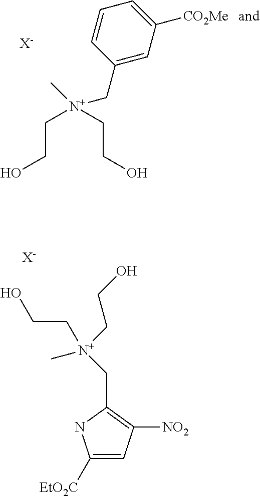

Among the novel compounds of formula (I), mention may be made of the compounds of formula (II) below, and also the optical isomers and/or geometrical isomers thereof:

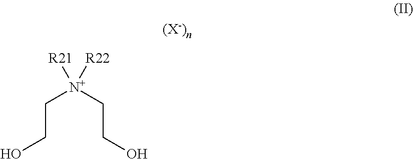

in which:

R21 denotes a linear or branched C₁-C₁₂ alkyl radical which is saturated or unsaturated with an ethylenic double bond;

R22 denotes a CₘH₂ₘAr aralkyl radical, where m is an integer ranging from 1 to 6 and Ar denotes an aryl radical chosen among phenyl, naphthyl, or furyl, substituted with a COR group, in particular a phenyl radical substituted with a COR group;

R has the same meaning previously indicated in formula (I);

n is equal to 0 or 1;

the electrical neutrality of said compounds being ensured by an external anion X⁻ (n=1) having the same meaning indicated previously in formula (I) or in the form of an internal salt (n=0) when a single COOH radical is present and forms a CO₂⁻ radical;

it being understood that when R1 denotes a methyl radical and R2 denotes a methyl radical substituted with a —COOH group, then n=1;

said compounds being other than the compounds having the following formulas:

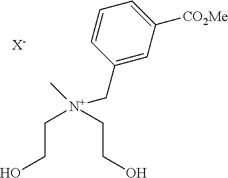 [SMILES: COC(=O)c1cccc(C[N+](C)(CCO)CCO)c1 ; "X-"]

Among the novel compounds of formula (I), mention may be made of the compounds of formula (II) below, and also the optical isomers and/or geometrical isomers thereof:

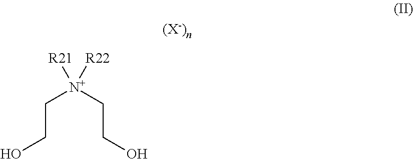

in which:

R21 denotes a linear or branched C₁-C₁₂ alkyl radical which is saturated or unsaturated with an ethylenic double bond;

R22 denotes a —(CH₂)ₚ-Ph radical substituted with a COR group with Ph denoting phenyl and p=1, 2, 3, or 4, in particular 1

R has the same meaning previously indicated in formula (I);

n is equal to 0 or 1;

the electrical neutrality of said compounds being ensured by an external anion X⁻ (n=1) having the same meaning indicated previously in formula (I) or in the form of an internal salt (n=0) when a single COOH radical is present and forms a CO₂⁻ radical;

it being understood that when R1 denotes a methyl radical and R2 denotes a methyl radical substituted with a —COOH group, then n=1;

said compounds being other than the compounds having the following formulas:

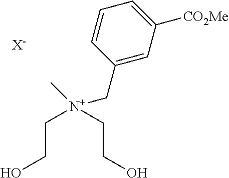 [SMILES: COC(=O)c1cccc(C[N+](C)(CCO)CCO)c1 ; "X-"]

As examples of compounds of formula (II), mention may be made of the compounds of formula (IIA) below, and also the optical isomers and/or geometrical isomers thereof:

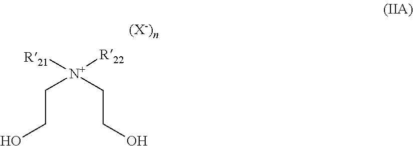

in which:

R′21 denotes a linear or branched, saturated C₁-C₁₂ alkyl radical, such as methyl;

R′22 denotes a —(CH₂)ₚ-Ph radical substituted with a COR group with Ph denoting phenyl and p=1, 2, 3, or 4, in particular 1;

R denotes a radical chosen from —OR′ and —NR′R″ in which R′ and R″ independently denote hydrogen or a linear or branched, saturated C₁-C₆ alkyl radical; in particular R denotes an ethoxy radical;

X⁻ has the same meaning indicated in formula (I).

n=0 or 1.

Among these compounds of formula (IIA), mention will in particular be made of the compounds (c) of formula:

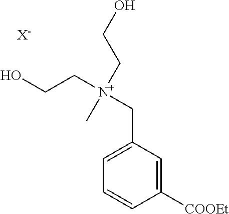

and quite particularly the compound (6) of formula:

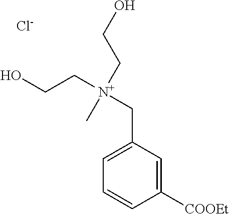 [SMILES: CCOC(=O)c1cccc(C[N+](C)(CCO)CCO)c1 ; [Cl-]]

Among the novel compounds of formula (I), mention may be made of the compounds of formula (III) below, and also the optical isomers and/or geometrical isomers thereof:

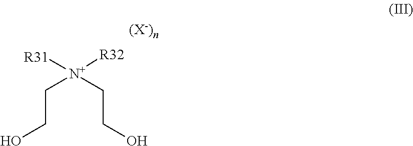

in which:

R31 and R32, chosen independently, denote:

(i) a linear or branched C₁-C₁₂ alkyl radical which is saturated or unsaturated (ethylenic double bond) and which is substituted with a COR group;

(ii) a CₘH₂ₘAr aralkyl radical, where m is an integer ranging from 1 to 6 and Ar is an aryl radical, in particular a phenyl radical, substituted with a COR group;

R has the same meaning previously indicated in formula (I);

n is equal to 0 or 1;

the electrical neutrality of said compounds being ensured by an external anion X⁻ (n=1) having the same meaning indicated previously in formula (I) or in the form of an internal salt (n=0) when a single COOH radical is present and forms a CO₂⁻ radical;

it being understood that when R1 denotes a methyl radical and R2 denotes a methyl radical substituted with a —COOH group, then n=1;

said compounds being other than the compounds having the following formula:

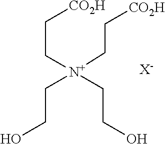

As examples of compounds of formula (III), mention may be made of the compounds of formula (IIIA), and also the optical isomers and/or geometrical isomers thereof:

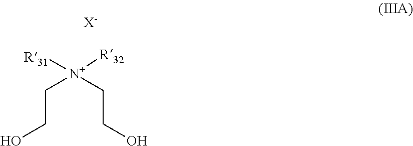

in which:

R′31 and R′32, independently, denote a linear or branched, saturated C₁-C₁₂ alkyl radical which is substituted with a COR group;

R denotes a radical chosen from —OR′ and —NR′R″ in which R′ and R″ independently denote a linear or branched, saturated C₁-C₆ alkyl radical; in particular R denotes an ethoxy radical;

X⁻ has the same meaning indicated in formula (I).

Among these compounds of formula (IIIA), mention will in particular be made of compounds (b) of formula:

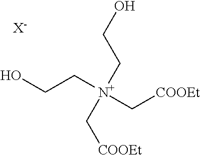 [SMILES: CCOC(=O)C[N+](CCO)(CCO)CC(=O)OCC ; "X-"]

and most particularly the compound (5) of formula:

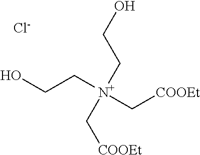 [SMILES: CCOC(=O)C[N+](CCO)(CCO)CC(=O)OCC ; [Cl-]]

A subject of the invention is also a composition comprising, in particular in a physiologically acceptable medium, one or more compounds of formula (II) and/or one or more compounds of formula (III) as defined previously.

In particular, a subject of the invention is also a composition comprising, especially in a physiologically acceptable medium, one or more compounds of formula (IIA) and/or one or more compounds of formula (IIIA) as defined previously.

More particularly, a subject of the invention is also a composition comprising, especially in a physiologically acceptable medium, one or more compounds of formula (c) and/or one or more compounds of formula (b) as defined previously, even more particularly a composition comprising, in particular in a physiologically acceptable medium, especially a cosmetically acceptable medium, the compound of formula (6) and/or the compound of formula (5) as defined previously.

### IV. Compositions

The compound(s) of formula (I) are preferably present in amounts ranging from 0.01% to 10% by weight, preferentially from 0.02% to 6% and even more particularly from 0.1% to 5% by weight, in particular from 2% to 7% by weight and more particularly from 3% to 6% by weight relative to the total weight of the composition.

As indicated previously, the invention also relates to a composition comprising one or more compounds of formula (I) as defined previously and one or more additional moisturizing active agents other than the compounds of formula (I).

Preferably, the additional moisturizing active agents are chosen from glycerol, urea, hydroxyethylurea, hyaluronic acid, propanediol, trehalose, mannitol, xylitol, sorbitol, glycine, β-alanine, taurine, trimethylglycine, and polyethylene glycol (PEG) derivatives.

Another subject of the invention is a composition comprising, in a physiologically acceptable medium, one or more compounds chosen from compounds (1), (2), (3), (4), (7), (8), (9), (10), (11), (13) and (14), and also the optical isomers and/or geometrical isomers thereof, said composition also possibly containing one or more additional moisturizers as defined previously, other than the compounds of formula (I).

The invention also relates to a composition characterized in that it comprises, especially in a physiologically acceptable medium, one or more compounds of formula (II) and/or one or more compounds of formula (III) as defined previously, said composition also possibly containing one or more additional moisturizers as defined previously, other than the compounds of formula (I).

In particular, the invention also relates to a composition characterized in that it comprises, especially in a physiologically acceptable medium, one or more compounds of formula (IIA) and/or one or more compounds of formula (IIIA) as defined previously, said composition also possibly containing one or more additional moisturizers as defined previously, other than the compounds of formula (I).

More particularly, the invention also relates to a composition characterized in that it comprises, especially in a physiologically acceptable medium, one or more compounds of formula (c) and/or one or more compounds of formula (b) as defined below, and even more particularly, the invention also relates to a composition characterized in that it comprises, especially in a physiologically acceptable medium, a compound of formula (6) and/or a compound of formula (5) as defined previously, said composition also possibly containing one or more additional moisturizers as defined previously, other than the compounds of formula (I).

### V. Treatment Process

The invention also relates to a cosmetic process for moisturizing keratin materials, in particular the skin, which consists in applying to a human keratin material, preferably to the skin, one of the compositions as defined previously.

Preferably, the keratin materials, such as the skin, are human keratin materials.

More particularly, the present invention relates to a cosmetic process for moisturizing dry skin, characterized in that one of the compositions as defined previously is applied to a dry skin.

Preferably, the cosmetic treatment process according to the present invention is intended to promote the persistence of a moisturizing treatment.

More particularly, the process of the invention, consisting in applying the compositions comprising the compound(s) of formula (I), preferably the compound(s) of formulae (II) and/or (III), especially promotes the persistence of a moisturizing treatment, and especially makes it possible to have satisfactory moisturizing activity after 6 hours, or even after 24 hours of a single application thereof to keratin materials, such as the skin.

More particularly, the process according to the invention is characterized in that said composition according to the invention comprising the compound(s) of formula (I), preferably the compound(s) of formulae (II) and/or (III), is applied once or repeated one to two times per day, preferably once a day, preferably over a period of at least one week, and more particularly of at least four weeks.

The cosmetic treatment process according to the present invention especially makes it possible to give the keratin material, preferably the skin, long-lasting moisturization after repeated application, i.e. the moisturizing effect persists for several days and/or one or more weeks after the end of application, even if the keratin materials, such as the skin, are no longer in contact with the compounds according to the invention or the compositions comprising them according to the invention.

The compound(s) of formula (I) according to the invention, or the compositions comprising them, may be used once or repeated one to two times per day, preferably once a day, preferably for at least one week, and more particularly for at least four weeks.

More particularly, the composition is applied to the keratin material, preferably the skin, i.e. on to a cutaneous region chosen from:
the hands,
the face, in particular the forehead, the cheeks or the contour of an eye (periocular), and in particular the crow's feet, the region below the eye (bag), or the eyelids,
the neck,
the feet,
the legs,
the arms and forearms.

### Galenical Forms

These compositions in which the compounds used according to the invention may be implemented are useful for the non-therapeutic care of the skin. They are in particular useful for moisturizing the skin, in particular for treating dry skin.

They may show their efficacy as non-therapeutic skin maintenance treatment, namely preventive treatment. They may also be used as non-therapeutic skin treatment after skin moisturization disorders have appeared.

A composition used according to the invention is advantageously suitable for topical application to the skin.

This composition may be a care composition, especially a dermatological composition. Preferably, it is a skincare composition.

For topical application to the skin, a composition according to the invention may be in any galenical form conventionally intended for this type of application and especially in the form of aqueous gels or aqueous or aqueous-alcoholic solutions. By adding a fatty or oily phase, they may also be provided in the form of dispersions of lotion type, of emulsions of liquid or semi-liquid consistency of milk type, obtained by dispersing a fatty phase in an aqueous phase (O/W) or conversely (W/O), or of suspensions or emulsions of soft, semi-solid or solid consistency of the cream or gel type, or alternatively of multiple emulsions (W/O/W or O/W/O), of microemulsions, of vesicular dispersions of ionic and/or nonionic type, or of wax/aqueous phase dispersions. These compositions are prepared according to the usual methods.

### Aqueous Phase

The compositions according to the invention intended for cosmetic use may comprise at least one aqueous phase. They are in particular formulated as aqueous lotions or as water-in-oil or oil-in-water emulsions or as multiple emulsions (oil-in-water-in-oil or water-in-oil-in-water triple emulsions (such emulsions are known and described, for example, by C. Fox in “Cosmetics and Toiletries”—November 1986—Vol. 101—pages 101-112)).

The aqueous phase of said compositions contains water and generally other water-soluble or water-miscible solvents. The water-soluble or water-miscible solvents comprise short-chain monoalcohols, for example of C₁-C₄, for instance ethanol or isopropanol; diols or polyols.

The compositions according to the invention preferably have a pH ranging from 3 to 9, depending on the chosen support.

When the composition(s) are in emulsion form, they generally contain, depending on the nature of the emulsion, one or more emulsifying surfactants.

The total amount of emulsifiers will preferably be, in the composition(s) according to the invention, in active material contents ranging from 1% to 8% by weight and more particularly from 2% to 6% by weight relative to the total weight of the composition.

### Fatty Phase

The compositions according to the invention may contain at least one water-immiscible organic liquid phase, known as a fatty phase. This phase generally comprises one or more hydrophobic compounds that make said phase water-immiscible. Said phase is liquid (in the absence of structuring agent) at room temperature (20-25° C.). Preferentially, the water-immiscible organic liquid phase in accordance with the invention generally comprises at least one volatile oil and/or one non-volatile oil and optionally at least one structuring agent.

The term “oil” means a fatty substance that is liquid at room temperature (25° C.) and atmospheric pressure (760 mmHg, i.e. 1.05×10⁵ Pa). The oil may be volatile or non-volatile.

For the purposes of the invention, the term “volatile oil” means an oil that is capable of evaporating on contact with the keratin material, such as the skin, in less than one hour, at room temperature and atmospheric pressure. The volatile oils of the invention are volatile cosmetic oils, which are liquid at room temperature, having a non-zero vapour pressure, at room temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

The term “non-volatile oil” means an oil that remains on the keratin material, such as the skin, at room temperature and atmospheric pressure for at least several hours and that especially has a vapour pressure of less than 10⁻³ mmHg (0.13 Pa).

The oil may be chosen from any physiologically acceptable oil and in particular cosmetically acceptable oil, especially mineral, animal, plant or synthetic oils; in particular volatile or non-volatile hydrocarbon-based oils and/or silicone oils and/or fluoro oils, and mixtures thereof.

More precisely, the term “hydrocarbon-based oil” means an oil mainly comprising carbon and hydrogen atoms and optionally one or more functions chosen from hydroxyl, ester, ether and carboxylic functions. Generally, the oil has a viscosity of from 0.5 to 100 000 mPa·s, preferably from 50 to 50 000 mPa·s and more preferably from 100 to 300 000 mPa·s.

As examples of volatile oils that may be used in the invention, mention may be made of:
volatile hydrocarbon-based oils chosen from hydrocarbon-based oils containing from 8 to 16 carbon atoms, and especially C8-C16 isoalkanes of petroleum origin (also known as isoparaffins).

As examples of non-volatile oils that may be used in the invention, mention may be made of:
hydrocarbon-based plant oils such as liquid triglycerides of fatty acids of 4 to 24 carbon atoms, for instance caprylic/capric acid triglycerides such as those sold by the company Stéarineries Dubois or those sold under the names Miglyol 810, 812 and 818 by the company Dynamit Nobel, and jojoba oil,
linear or branched hydrocarbons, of mineral or synthetic origin, such as liquid paraffins and derivatives thereof, petroleum jelly, polydecenes, polybutenes, hydrogenated polyisobutene such as Parleam, or squalane;
synthetic ethers containing from 10 to 40 carbon atoms;
synthetic esters such as isononyl isononanoate, isopropyl myristate, isopropyl palmitate or C12 to C15 alkyl benzoates;
silicone oils such as linear (dimethicone) or cyclic (cyclomethicone) non-volatile polydimethylsiloxanes (PDMSs).

The compositions according to the invention may also comprise one or more cosmetic adjuvants chosen from softeners, opacifiers, stabilizers, preserving agents, fragrances, a structuring agent for a fatty phase, in particular chosen from waxes, pasty compounds, gelling agents; organic or mineral fillers; thickeners or suspending agents, or any other ingredient normally used in cosmetics for this type of application.

Needless to say, those skilled in the art will take care to select this or these optional additional compounds such that the advantageous properties intrinsically associated with the composition in accordance with the invention are not, or are not substantially, adversely affected by the envisaged addition(s).

The examples below illustrate the invention without, however, limiting the scope thereof.

### EXAMPLES

### A) Synthesis Examples

### Example 1: Synthesis of Compound (4)

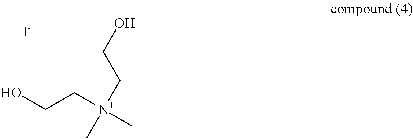

The synthesis of compound (4) is described in the following article: El-Sayed, A. A., Kantouch, F. A. and Kantouch, A. (2011), Preparation of cationic polyurethane and its application to acrylic fabrics. J. Appl. Polym. Sci., 121: 777-783

### Example 2: Synthesis of Compound (12)

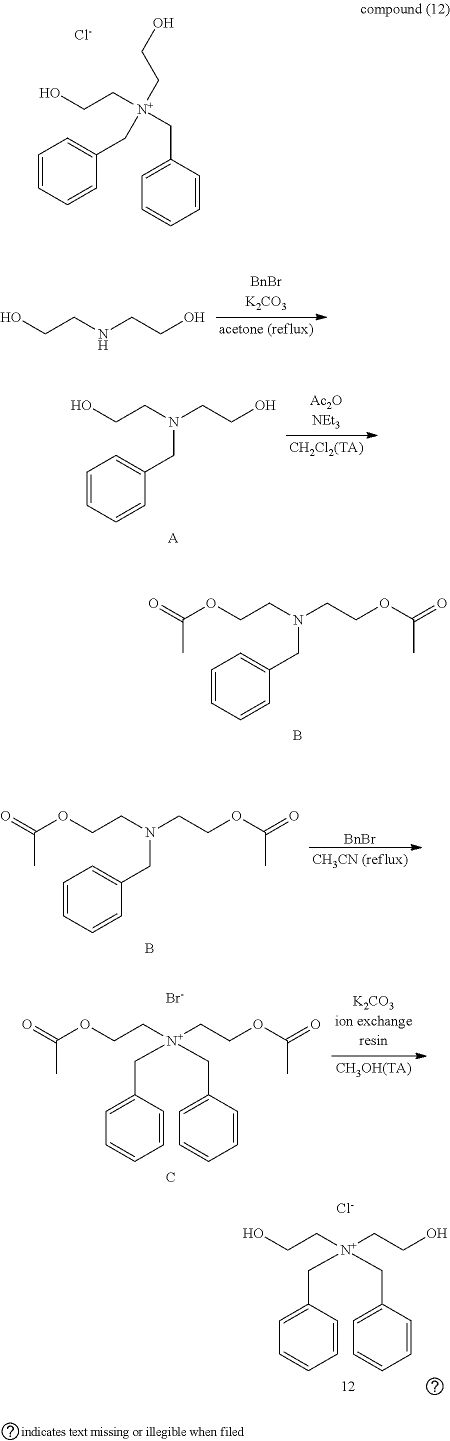

### Synthesis of Intermediate A:

Potassium carbonate (60 g, 435 mmol) was suspended in acetone (500 ml). Diethanolamine (21 g, 200 mmol) and benzyl bromide (34.4 g, 200 mmol) were successively added. The reaction mixture was refluxed for 8 hours, cooled to room temperature and filtered. The filtrate was concentrated to dryness and then the residue was purified by silica gel column chromatography (dichloromethane/methanol: 10/1) to isolate product A in the form of a colourless oil (37.5 g, 75% yield).

### Synthesis of Intermediate B:

Product A (37.2 g, 190 mmol) was dissolved in dichloromethane (300 ml). Acetic anhydride (46.5 g, 456 mmol) and triethylamine (164 ml, 1.3 mol) were successively added. The reaction mixture was stirred at room temperature for 3 hours and filtered. The filtrate was concentrated to dryness and the residue was then purified by silica gel column chromatography (hexane/ethyl acetate: 20/1) to isolate product B in the form of a yellow oil (24 g, 45% yield).

### Synthesis of Intermediate C:

A solution of product B (24 g, 86 mmol) and of benzyl bromide (88 g, 514 mmol) in acetonitrile (500 ml) was refluxed for 16 hours. The reaction mixture was then concentrated under reduced pressure and the residue was purified by silica gel column chromatography (dichloromethane/methanol: 30/1) to isolate product C in the form of a colourless oil (2.7 g, 7% yield).

### Synthesis of Compound (12)

A mixture of the intermediate C (2.7 g, 6 mmol) and of potassium carbonate (1.2 g, 8.7 mmol) in methanol was stirred at room temperature for 15 hours. IR-120 ion exchange resin (12 g), pre-rinsed with absolute methanol, was added. The suspension was stirred for 30 minutes and then filtered. The filtrate was concentrated under reduced pressure so as to produce a white solid. Since the Br⁻/Cl⁻ interconversion was not complete, the product was again dissolved in methanol and treated with Dowex 1x2-200 ion exchange resin (5 g). The suspension was stirred at room temperature for 15 hours and then filtered to remove the resin. Dowex 1x2-200 resin (5 g) was again added and the suspension was stirred at room temperature for 15 hours. The Dowex resin treatment cycle was repeated a further 5 times. After the final filtration, the solvent was evaporated off and the expected product was isolated in the form of a white solid (1.2 g, 61% yield).

### Example 3: Synthesis of Compound (7)

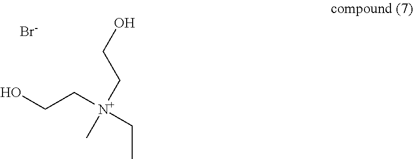

N,N-Diethanolamine (2.38 g, 20 mmol) was dissolved in methanol (100 ml). 2-Bromoethane (21.6 g, 200 mmol, 10 equivalents) was added dropwise. The reaction mixture was stirred at room temperature for 15 hours and then concentrated to dryness. The residue was purified by recrystallization from a methanol/acetone mixture and compound (7) was obtained in the form of a white solid (3.2 g, 70% yield).

### Example 4: Synthesis of Compound (8)

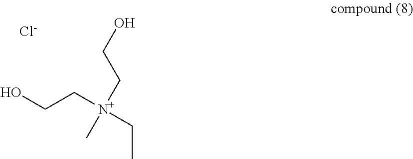

Compound (7) (3 g, 13.2 mmol) was dissolved in 50 ml of a methanol/water mixture (5/1). An ion exchange resin (ref. Dowex 1x2-200, 10 g) was suspended, the mixture was stirred at room temperature for 5 hours and the resin was then filtered off. The process was repeated until the Br⁻ ion content reached 0.5%. The solvent was then evaporated off under vacuum and compound (8) was isolated in the form of a white solid (2.1 g, 70% yield). The ¹H NMR spectrum (DMSO-d6) and the mass spectrum confirmed the structure of the expected product (8).

### Example 5: Synthesis of Compound (9)

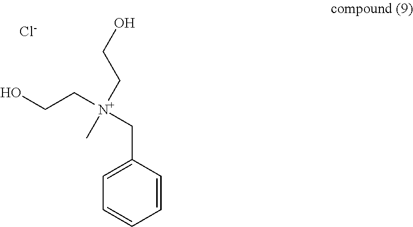

N-Methyl-N,N-diethanolamine (1.78 g, 15 mmol) was dissolved in methanol (60 ml). Benzyl chloride (9.5 g, 75 mmol, 5 equivalents) was added dropwise and the reaction mixture was refluxed for 15 hours. The solvent was then evaporated off under reduced pressure and the residue was purified by silica gel column chromatography (dichloromethane/methanol: 10/1) to obtain compound (9) in the form of a white solid (1.6 g, 44% yield). The ¹H NMR spectrum (DMSO-d6) and the mass spectrum confirmed the structure of the expected product (9).

### Example 6: Synthesis of Compound (10)

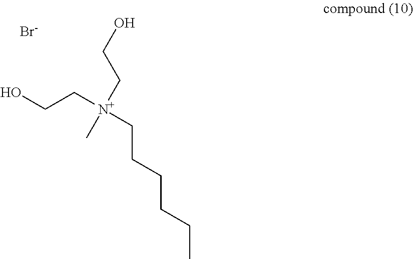

N-Methyl-N,N-diethanolamine (2 g, 17 mmol) was dissolved in methanol (20 ml). 1-Bromohexane (13.9 g, 84 mmol, 5 equivalents) was added dropwise and the reaction mixture was refluxed for 8 hours. The solvent was then evaporated off under reduced pressure and the residue was purified by silica gel column chromatography (dichloromethane/methanol: 20/1) to obtain compound (10) in the form of a colourless oil (2.3 g, 48% yield).

### Example 7: Synthesis of Compound (11)

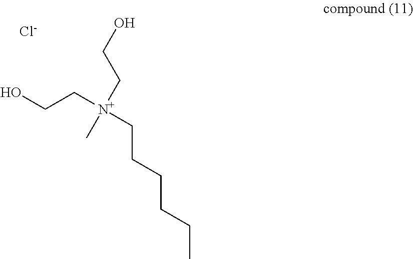

Compound (10) (2.3 g, 8.1 mmol) was dissolved in 20 ml of methanol. An ion exchange resin (ref. Dowex 1x2-200, 5 g) was suspended, the mixture was stirred at room temperature for 0.15 hours and the resin was then filtered off. The process was repeated 5 times. The solvent was then evaporated off under vacuum and compound (11) was isolated in the form of a slightly yellow oil (1.6 g, 82% yield). The ¹H NMR spectrum (DMSO-d6) and the mass spectrum confirmed the structure of the expected product (11).

### Example 8: X— Interchange

The different salts are prepared the following way starting for example from the X═Cl or X=MeSO3 using resins Interchange as described in the figure below.

Empty cartridge was filled with hydrogenocarbonate resin (commercially available from Agilent Technology under reference PL3540-4603). Once the cartridge packed, resin was washed with demineralized water (100 ml water for 20 g of resin).

Compound 4 X—=MeSO3- was solubilized in water at the concentration of 0.5 mol/L and adsorbed onto the column by gravity. Resin was then washed with water 3 times dead volume with pH control to desorb the intermediate into a stock solution.

The desired new carboxylate anion is then introduced under its acidic form (RCOOH) into the stock solution and CO2 is gently bubbling showing interchange. If the carboxylic acid is not soluble in water, tetrahydrofuran can be added to solubilize it.

The solution is then concentrated and freeze dried.

Compounds according to the invention is then characterized by NMR (disappearance of MeSO3-), MS and elemental analysis.

| | Compounds | | | |
|---|---|---|---|---|
| | as listed in | | | QC: |
| | the table | | | RMN 1H/ |
| Acid | below | Yield | Aspect | MS |
| 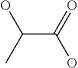 | 13 | Quanitiative | colorless oil | conform |
| 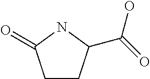 | 14 | Quanitiative | colorless oil | conform |
| 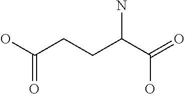 | 15 | Quanitiative | opalescent oil | conform |
| 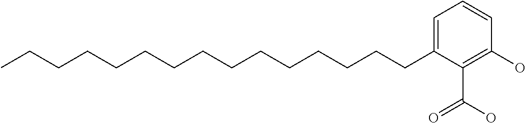 [SMILES: CCCCCCCCCCCCCCCc1cccc(O)c1C(=O)O] | 16 | 94 | grey solid | conform |
| 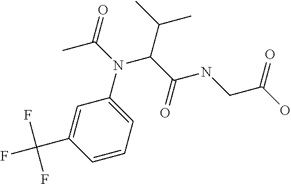 [SMILES: CC(=O)N(c1cccc(C(F)(F)F)c1)C(C(=O)NCC(=O)O)C(C)C] | 17 | 95 | white solid | conform |
| 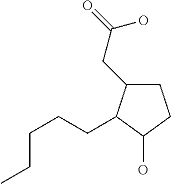 | 18 | 97 | yellow thick oil | conform |
| 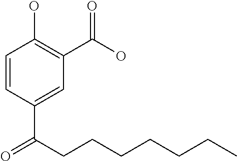 [SMILES: CCCCCCCC(=O)c1ccc(O)c(C(=O)O)c1] | 19 | Quantitative | yellow light solid | conform |
| 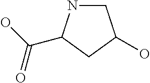 | 20 | Quantitative | white paste | conform |
| 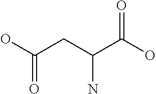 | 21 | Quantitative | yellow thick oil | conform |
| 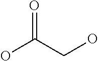 | 22 | Quantitative | colorless oil | conform |
| 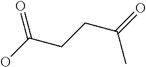 | 23 | Quantitative | colorless oil | conform |
| 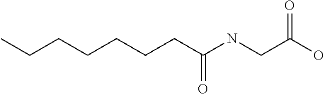 | 24 | 95 | white paste | conform |

### Compounds:

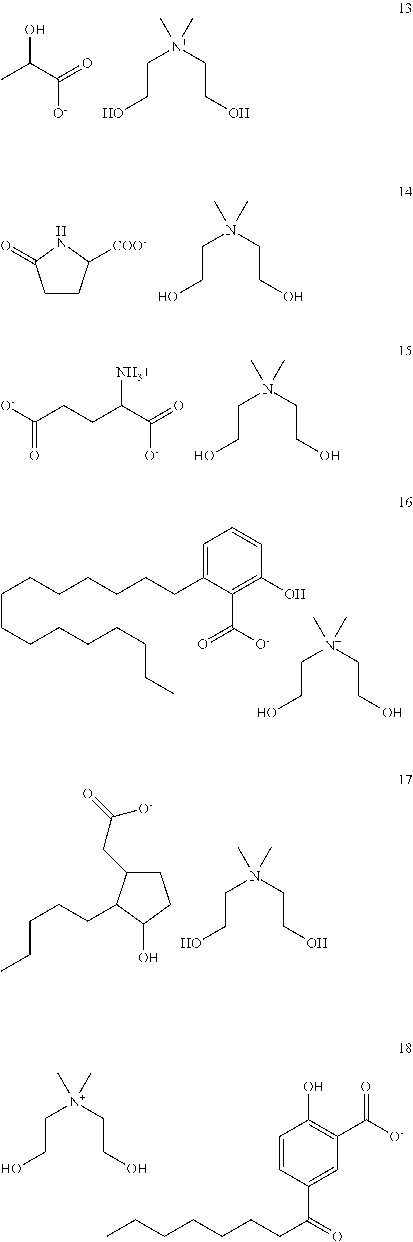

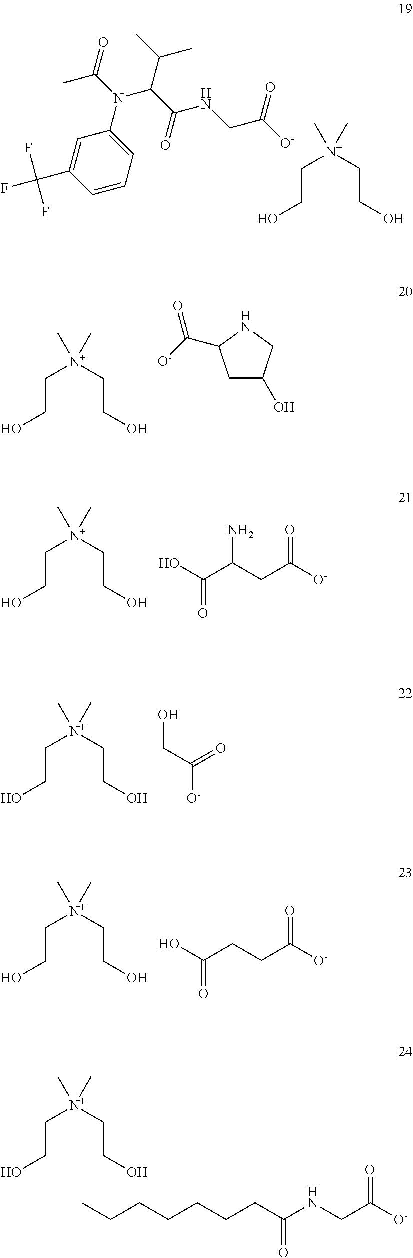

### B): Evaluation of the Moisturizing Potential on Isolated Stratum Corneum by Measurement with a Corneometer

A test was performed to evaluate the moisturizing potential of the compounds of the invention formulated in an aqueous solution to an amount of 5% by weight relative to the total weight of the composition.

The technique makes it possible to measure the dielectric capacitance of stratum corneum (SC), which depends on the mean dielectric permittivity value of the tissue. The dielectric permittivity varies greatly with the amount of water contained in the SC.

The SC samples are conditioned at 75% relative humidity and at 25° C. before/during the measurements and the treatment. The capacitance measurement is performed using a Corneometer™ (Courage & Khazaka, Germany).

The test compound, compound 2 according to the invention or a moisturizing active agent such as glycerol, is dissolved in a water/n-propanol mixture (80/20) and the solution is deposited onto the SC at a rate of 10 μl/cm² followed by air-drying for a total duration of 1 hour 30 minutes.

Measurement is taken at T0, before the treatment, and a measurement Ttreat is taken after total drying of the treatment.

Each treatment is systematically compared with its control (vehicle) and with its T0.

Using at least two different batches of SC, four to five SC samples are measured per treatment.

The variation in the corneometer signal (HCM) after treatment is calculated first for each SC sample: DHCMi=HCMi(Ttreat)−HCMi(T0). The mean of the DHCMi(vehicle) variations is then calculated for the control samples (treated with the vehicle); this mean value is subtracted from all the DHCMi(active agent) variations to correct the systematic bias.

The following is measured for each sample i:
For the vehicle (control): DHCMi(veh)=HCMiᵥₑₕ(Ttreat)−HCMiᵥₑₕ(T0)
For the active agent: DHCMi_{active agent}=HCMi_{active agent}(Ttreat)−HCMi_{active agent}(T0)

To correct the systematic bias associated with the vehicle, the corrected value DHCMi_{corr. active agent} is considered for the active agent according to: DHCMi_{corr. active agent}−DHCMi_{active agent}−M

in which M corresponds to the mean of the DHCMi(veh) variations observed on the n control samples:

$$M = \frac{1}{n} {\sum }_{i = 1}^{n} DHCMi ( veh )$$

The mean DHCMi corr. active agent values, and also the associated standard deviations that were obtained, are reported in the table below for compound 2 tested at 5%, in comparison with glycerol at 5%:

| | | Compound 2 |
|---|---|---|
| | Glycerol | according to |
| Products tested in a | at 5% | the invention |
| water/n-propanol mixture | by weight | at 5% by weight |
| Mean DHCMi corr. active agent | 13.8 | 25.9 |
| Standard deviation | 5.1 | 6.6 |

The above results are obtained from two measurement runs (more than 4 samples)

The results presented above were obtained over two measurement runs on two batches of stratum corneum. The response of the products is analysed in each run relative to the vehicle.

It emerges that this test shows that the dielectric capacitance of the stratum corneum (SC) is much better with compound 2 used according to the invention in comparison with that obtained with glycerol at the same concentration.

Unexpectedly, compound 2 used according to the invention allows good moisturization of the stratum corneum and thus of the skin. This moisturizing effect proves to be greater than that of glycerol.

The mean DHCMi corr. active agent values, and also the associated standard deviations that were obtained, are reported in the table below for two salts of compound a, compound 13 and compound 14 tested at 5%, in comparison with glycerol at 5%:

| Products tested | | Compound 13 | Compound 14 |
|---|---|---|---|
| in a | Glycerol | according to | according to |
| water/n-propanol | at 5% | the invention | the invention |
| mixture | by weight | at 5% by weight | at 5% by weight |
| Mean DHCMi corr. | 11 | 14 | 13 |
| active agent | | | |

The results presented above were obtained over two measurement runs on two batches of stratum corneum. The response of the products is analysed in each run relative to the vehicle.

It emerges that this test shows that the dielectric capacitance of the stratum corneum (SC) is better with the compounds 13 and 14 used according to the invention in comparison with that obtained with glycerol at the same concentration.

Unexpectedly, these 2 compound 13 and 14 used according to the invention allows good moisturization of the stratum corneum and thus of the skin.

### C): Evaluation of the Pleasant Feel of the Compositions, i.e Having No Discomfort Sensations Such as Tautness by Measurement with Internal Stresses Test

The aim of this study is to compare the properties of the deposit formed when applying aqueous solutions of raw material HYBRIDUR 875 ® available from AIR PRODUCTS AND CHEMICALS (which is a an aqueous 40% dispersion of an interpenetrated networks of polyurethane polymers and polyacrylic). This raw material is currently used in skincare products present on the market. It is well known for its tightening properties but also for the discomfort generated Hybridur 875® solution forms a deposit which develops high values of internal stresses, as already well-known.

The principle of internal stress test is representative of generated tightness.

The solutions of polymer are deposited on a nitrile substrate cut in 12 specimens (see below). A given volume is applied on each specimen, with 4 replicates by solution.

For standard test, a volume of 26 μl of a solution of 7% of active material is applied. It corresponds to a quantity of 0.64 mg·cm⁻² of active material on the substrate after drying. The volume or concentration of solutions may vary with or without variations of the final quantity per cm² after drying. In such case this will be specified.

Measurements are realized, in standard conditions, after 3 and/or 24 hours of drying at 25° C. at 45% Relative Humidity (RH). These conditions may vary (times and rate of RH) and this is specified in such case.

The tightening effect is measured by image analysis. The surface of the specimen is determined before and after the deposit formation.

The % of internal stresses (contraintes internes in French CI) is calculated as:

CI=(1−Sᵢₙₜ/Sₜₒₜ)×100

### Where:

Sᵢₙₜ: internal surface of the specimen, comprised inside the retracted edges of the specimen.

Sₜₒₜ: total surface of the specimen before deposit of the solution

Two compositions has been prepared and tested:

Composition Y:—Solution of Hybridur 87® 5 at 16.8% by weight of raw material (7% of active material MA)+water 83.2% by weight compared to the total weight of the composition

Composition Z:—Solution of Hybridur 875® at 16.8% by weight+5% by weight of compound 2 (5% of active material MA) according to the invention+water 78.2%

Deposits for completion of the internal stress test were prepared on nitrile substrates. 26 μL solution (dispersion) are deposited and left to dry 3 hours before measurements. Deposits represent 0.6 mg·cm-2 after drying at 25° C. and 45% RH.

The measurements were performed after 3 hours of drying. Each measurement is repeated 4 times. In the case of the solution Hybridur 875® two sets of 4 measurements were performed.

The internal stress values obtained for deposits made from each of the two solutions measured after drying 3H are and reported in Table below.

| | internal stresses (%) | internal stresses (%) |
|---|---|---|
| Compositions | drying 3H | drying3H |
| Composition Y | 32 ± 3 | 35 ± 3 |
| Composition Z | 0.5 ± 0.7 | 0.9 ± 0.6 |

The Hybridur 875® solution, composition Y, forms a high deposition of internal stresses

The solution Hybridur 875®+5% of compound 2 according to the invention, composition Z, form a deposit that no longer develops internal stresses in the experimental accuracy close. Therefore, this deposit will not generate any sensation of tightness.

The addition of the compound 2 according to the invention allows to remove the feeling of discomfort generated by Hybridur 875® at high concentrations.

The composition comprises compounds according to the invention, such as compound 2, when applied on the keratin materials such as the skin have a pleasant feel for the consumers, i.e have no discomfort sensations such as tautness.

### D) Example 9: Skin Care Cream

| | Weight % |
|---|---|
| Compound 2 | 4% |
| Glyceryl monostearate | 0.8% |
| Cetyl alcohol | 2.0% |
| Stearyl alcohol | 5.0% |
| Polyoxyethylene (20 OE) stearate | 3.0% |
| Crosslinked acrylic acid (Carbopol 941) | 0.3% |
| Caprylic/capric triglycerides | 12.0% |
| Preserving agents qs | |
| Water qs | 100.0% |

When applied to the skin, the illustrated cosmetic formulation shows a good moisturizing effect on the skin.

1. A cosmetic process for moisturizing keratin materials which comprises applying to the keratin materials one or more diethanolammonium derivatives corresponding to formula (I) below, optical isomers and/or geometrical isomers thereof: 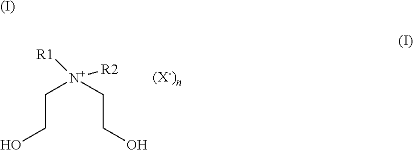
in which
R1 and R2 independently denote:
(i) a linear or branched C1-C12 alkyl radical which is saturated or unsaturated with one or more ethylenic double bond(s); said radical being optionally substituted with a group —COR; or
(ii) an aralkyl radical CₘH₂ₘAr such that m denotes an integer ranging from 1 to 6 and Ar denotes an aryl radical optionally substituted with a group —COR;
R denotes a group —OR′ or —NR′R″ where R′ and R″ independently denote hydrogen or a linear or branched C1-C6 alkyl radical which is saturated or unsaturated with an ethylenic double bond;
n is equal to 0 or 1;
the electrical neutrality of said compounds being provided by an organic or inorganic external anion X— (n=1) or internally (n=0) when a single COOH radical is present and forms a CO₂- radical;
it being understood that when R1 denotes a methyl radical and R2 denotes a methyl radical substituted with a —COOH group, then n=1.

2. The process according to claim 1, in which X⁻ is chosen from the anionic forms of organic compounds bearing one or more acid functions, such as the anionic forms of amino acids, methanesulfonate, para-toluenesulfonate, camphorsulfonate, tartrate, citrate, acetate, levulinate, or methosulfate, halides, sulfates, phosphates, hydrogen phosphates, dihydrogen phosphates, carbonate or hydrogen carbonate.

3. The process according to claim 1, in which X⁻ is chosen from the anionic forms of organic compounds bearing one or more carboxylic acid functions such as among compounds of formula A or B or C: 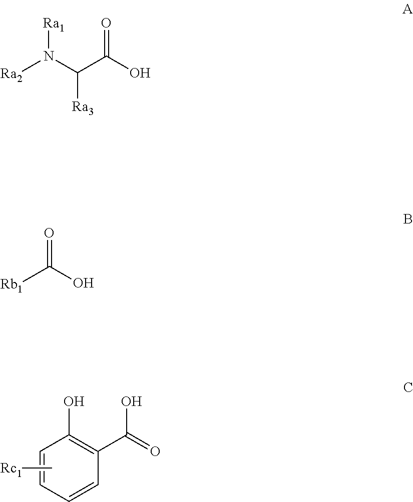
With
Ra1 means a hydrogen atom or an a linear or branched alkyl radical C1-C18
Ra2 means a hydrogen atom or an a linear or branched alkyl radical C1-C18 or an a linear or branched alkylcarbonyl radical C1-C18
Ra1 and Ra2 can together form a five to eight membered ring with the N atom bearing them.
Ra2 and Ra3 can together form a five membered ring of formula A1 or A2 with the N atom bearing them 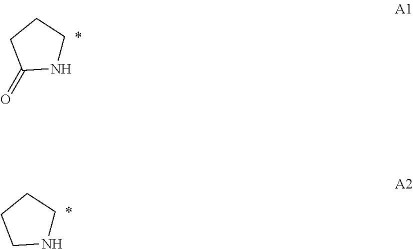 [SMILES: O=C1CCCN1 ; C1CCNC1]
Ra3 is chosen among the radicals described below

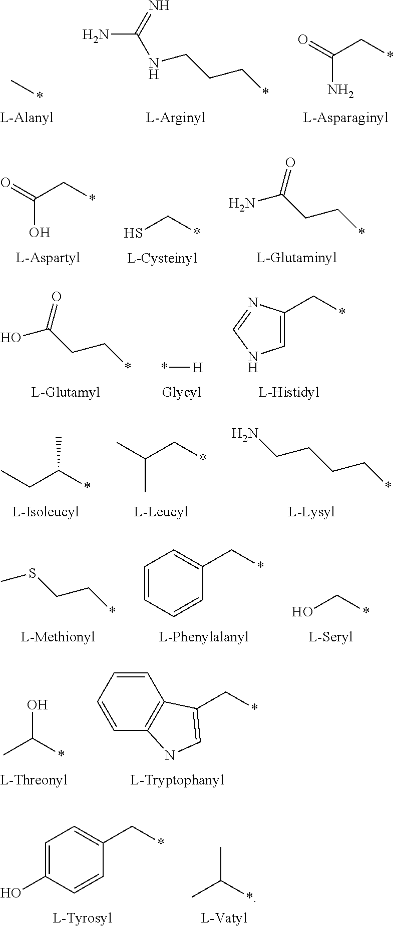

4. The process according to claim 1, in which the one or more diethanolammonium derivatives of formula (I) are chosen from the following compounds:

| Compounds | Structure |
|---|---|
| a | 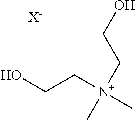 |
| b | 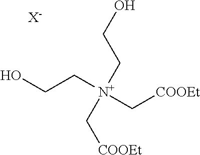 [SMILES: CCOC(=O)C[N+](CCO)(CCO)CC(=O)OCC ; "X-"] |
| c | 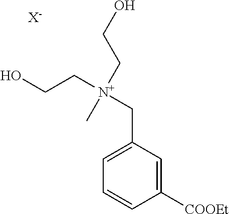 [SMILES: CCOC(=O)c1cccc(C[N+](C)(CCO)CCO)c1 ; "X-"] |
| d | 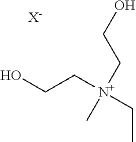 |
| e | 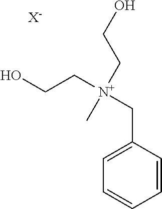 |
| f | 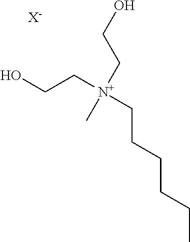 |
| g | 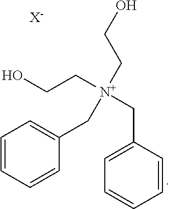 [SMILES: OCC[N+](CCO)(Cc1ccccc1)Cc1ccccc1 ; "X-"] |

5. The process according to claim 1, in which the one or more diethanolammonium derivatives of formula (I) are chosen from the following compounds:

| Compound | Structure |
|---|---|
| 1 | 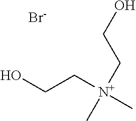 |
| 2 | 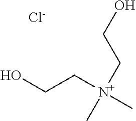 |
| 3 | 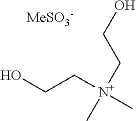 |
| 4 | 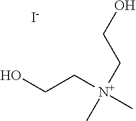 |
| 5 | 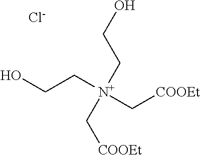 [SMILES: CCOC(=O)C[N+](CCO)(CCO)CC(=O)OCC ; [Cl-]] |
| 6 | 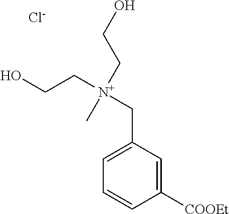 [SMILES: CCOC(=O)c1cccc(C[N+](C)(CCO)CCO)c1 ; [Cl-]] |
| 7 | 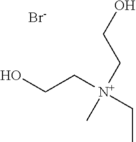 |
| 8 | 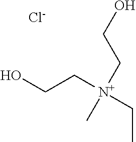 |
| 9 | 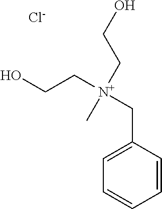 |
| 10 | 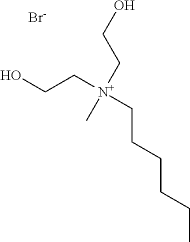 |
| 11 | 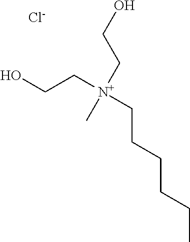 |
| 12 | 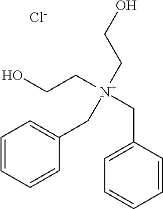 [SMILES: OCC[N+](CCO)(Cc1ccccc1)Cc1ccccc1 ; [Cl-]] |
| 13 | 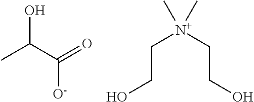 |
| 14 | 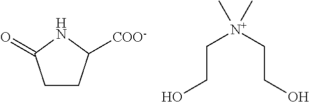 |
| 15 | 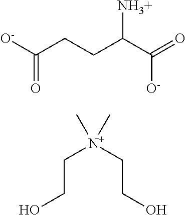 |
| 16 | 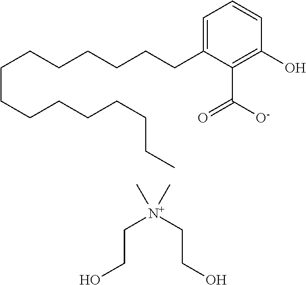 |
| 17 | 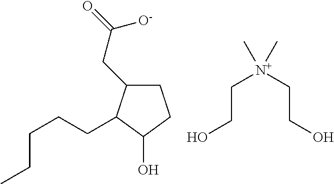 |
| 18 | 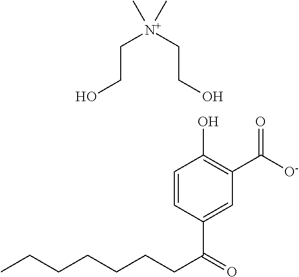 |
| 19 | 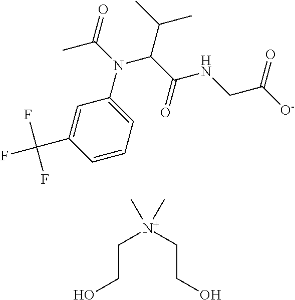 |
| 20 | 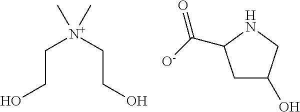 |
| 21 | 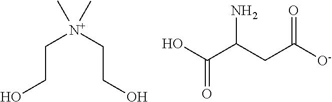 |
| 22 | 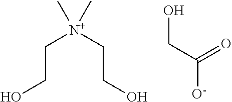 |
| 23 | 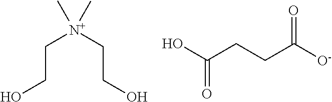 |
| 24 | 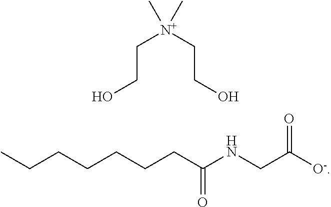 |

6. The process according to claim 1, in which the one or more diethanolammonium derivatives of formula (I) are such that R1 and R2 independently denote a linear or branched C₁-C₈ alkyl radical which is saturated or unsaturated with one or more ethylenic double bond(s), and which is optionally substituted with a group —COR.

7. The process according to claim 6, in which the one or more diethanolammonium derivatives of formula (I) are such that R1 and R2 independently denote a linear or branched, saturated C₁-C₈ alkyl radical which is optionally substituted with a group —COR where R denotes a radical chosen from —OR′ and —NR′R″, with R′ and R″ independently denoting hydrogen or a linear or branched, saturated C₁-C₆ alkyl radical.

8. The process according to claim 6, in which the one or more diethanolammonium derivatives of formula (I) are chosen from compounds (a), (b), (d) and (f).

9. A compound of formula (II) below, optical isomers and/or geometrical isomers thereof: 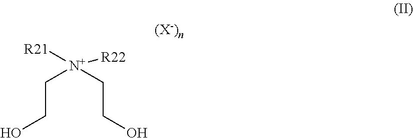
in which:
R21 denotes a linear or branched C₁-C₁₂ alkyl radical which is saturated or unsaturated with an ethylenic double bond;
R22 denotes a CₘH₂ₘAr aralkyl radical, where m is an integer ranging from 1 to 6 and Ar is an aryl radical substituted with a COR group, in particular a phenyl radical substituted with a COR group;
R denotes an —OR′ or —NR′R″ group where R′ and R″ independently denote hydrogen or a linear or branched C₁-C₆ alkyl radical which is saturated or unsaturated with an ethylenic double bond;
n is equal to 0 or 1;
the electrical neutrality of said compound being ensured by an organic or inorganic external anion X⁻ (n=1) or in the form of an internal salt (n=0) when a single COOH radical is present and forms a CO₂⁻ radical;
it being understood that when R1 denotes a methyl radical and R2 denotes a methyl radical substituted with a —COOH group, then n=1;
and said compounds being other than the compounds having the following formulas:

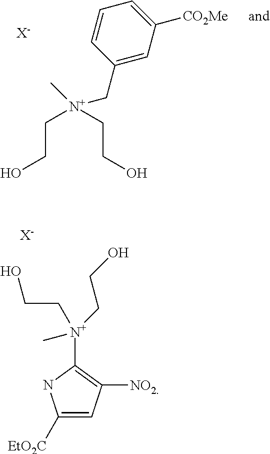

10. The compound according to claim 9, wherein R22 denotes a CₘH₂ₘAr aralkyl radical, where m is an integer ranging from 1 to 6 and Ar is an aryl radical substituted with a COR group chosen among phenyl naphtyl or furyl.

11. The compound according to claim 9, which is chosen from the compounds of formula (IIA) below, optical isomers and/or geometrical isomers thereof: 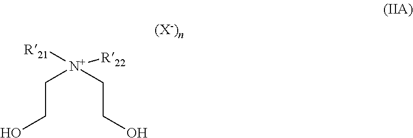
in which:
R′21 denotes a linear or branched, saturated C₁-C₁₂ alkyl radical, such as methyl;
R′22 denotes a —(CH₂)ₚ-Ph radical substituted with a COR group with Ph denoting phenyl and p is an integer ranging from 1 to 4;
R denotes a radical chosen from —OR′ and —NR′R″ in which R′ and R″ independently denote hydrogen or a linear or branched, saturated C₁-C₆ alkyl radical;
n is 0 or 1.

12. The compound according to claim 9 wherein which is chosen from compounds (c) of formula:

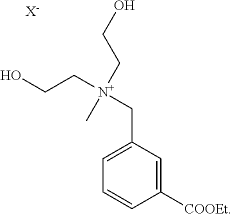 [SMILES: CCOC(=O)c1cccc(C[N+](C)(CCO)CCO)c1 ; "X-"]

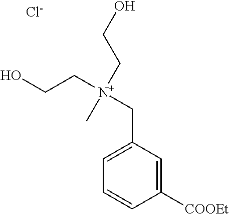 [SMILES: CCOC(=O)c1cccc(C[N+](C)(CCO)CCO)c1 ; [Cl-]]

13. A compound of formula (III) below, optical isomers and/or geometrical isomers thereof: 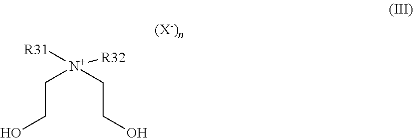
in which:
R31 and R32, chosen independently, denote:
(i) a linear or branched C₁-C₁₂ alkyl radical which is saturated or unsaturated (ethylenic double bond) and which is substituted with a COR group;
(ii) a CₘH₂ₘAr aralkyl radical, where m is an integer ranging from 1 to 6 and Ar is an aryl radical;
R denotes a group —OR′ or —NR′R″ where R′ and R″ independently denote hydrogen or a linear or branched C1-C6 alkyl radical which is saturated or unsaturated with an ethylenic double bond;
n is equal to 0 or 1;
the electrical neutrality of said compound being ensured by an organic or inorganic external anion X⁻ (n=1) or in the form of an internal salt (n=0) when a single COOH radical is present and forms a CO₂⁻ radical;
it being understood that when R1 denotes a methyl radical and R2 denotes a methyl radical substituted with a —COOH group, then n=1;
and said compound being other than the compounds having the following formula:

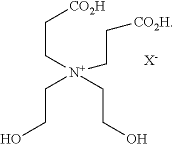

14. The compound according to claim 13, which is chosen from the compounds of formula (IIIA), optical isomers and/or geometrical isomers thereof: 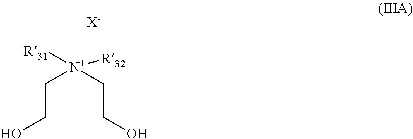
in which:
R′31 and R′32, independently, denote a linear or branched, saturated C₁-C₁₂ alkyl radical which is substituted with a COR group;
R denotes a radical chosen from —OR′ and —NR′R″ where R′ and R″ independently denote a linear or branched, saturated C₁-C₆ alkyl radical.

15. The compound according to claim 13, which is chosen from compounds (b) of formula: 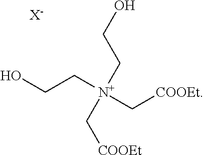 [SMILES: CCOC(=O)C[N+](CCO)(CCO)CC(=O)OCC ; "X-"]
and most particularly compound (5) of formula:

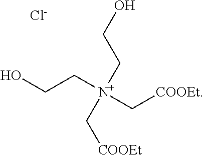 [SMILES: CCOC(=O)C[N+](CCO)(CCO)CC(=O)OCC ; [Cl-]]

16. A composition comprising one or more compounds of formula (I) as defined below:
optical isomers and/or geometrical isomers thereof: 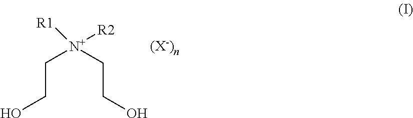
in which
R1 and R2 independently denote:
(i) a linear or branched C1-C12 alkyl radical which is saturated or unsaturated with one or more ethylenic double bond(s); said radical being optionally substituted with a group —COR; or
(ii) an aralkyl radical CₘH₂ₘAr such that m denotes an integer ranging from 1 to 6 and Ar denotes an aryl radical optionally substituted with a group —COR;
R denotes a group —OR′ or —NR′R″ where R′ and R″ independently denote hydrogen or a linear or branched C1-C6 alkyl radical which is saturated or unsaturated with an ethylenic double bond;
n is equal to 0 or 1;
the electrical neutrality of said compounds being provided by an organic or inorganic external anion X— (n=1) or internally (n=0) when a single COOH radical is present and forms a CO₂- radical;
it being understood that when R1 denotes a methyl radical and R2 denotes a methyl radical substituted with a —COOH group, then n=1;
and one or more additional moisturizing active agents other than the compounds of formula (I).

17. The composition according to claim 16, in which the compound(s) of formula (I) are present in amounts ranging from 0.01% to 10% by weight relative to the total weight of the composition.

18. Composition comprising, in a physiologically acceptable medium, in particular a cosmetically acceptable medium, one or more compounds chosen from:

| Compounds | Structure |
|---|---|
| d | 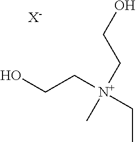 |
| e | 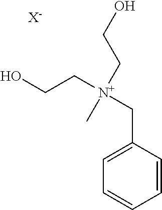 |
| f | 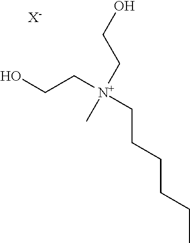 |
| g | 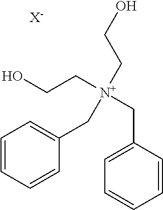 [SMILES: OCC[N+](CCO)(Cc1ccccc1)Cc1ccccc1 ; "X-"] |

wherein for compound (e), X⁻ is other than OH.

19. A composition comprising, in a physiologically acceptable medium one or more compounds chosen from the following compounds:

| Compound | Structure |
|---|---|
| 1 | 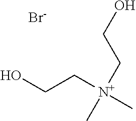 |
| 2 | 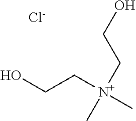 |
| 3 | 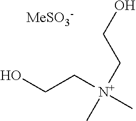 |
| 4 | 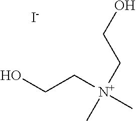 |
| 7 | 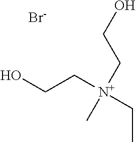 |
| 8 | 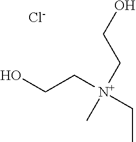 |
| 9 | 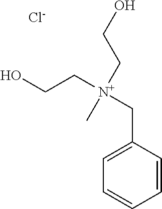 |
| 10 | 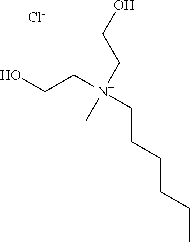 |
| 11 | 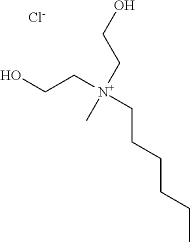 |
| 13 | 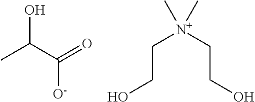 |
| 14 | 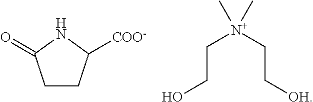 |

20. A composition, which comprises, in a physiologically acceptable medium, one or more compounds of formula (II) as defined in claim 9, optical isomers and/or geometrical isomers thereof and/or one or more compounds of formula (III) as defined below:
optical isomers and/or geometrical isomers thereof: 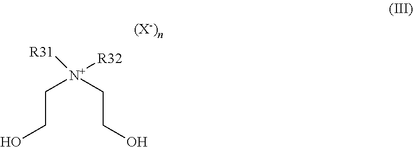
in which:
R31 and R32, chosen independently, denote:
(i) a linear or branched C₁-C₁₂ alkyl radical which is saturated or unsaturated (ethylenic double bond) and which is substituted with a COR group;
(ii) a CₘH₂ₘAr aralkyl radical, where m is an integer ranging from 1 to 6 and Ar is an aryl radical;
R denotes a group —OR′ or —NR′R″ where R′ and R″ independently denote hydrogen or a linear or branched C1-C6 alkyl radical which is saturated or unsaturated with an ethylenic double bond;
n is equal to 0 or 1;
the electrical neutrality of said compound being ensured by an organic or inorganic external anion X⁻ (n=1) or in the form of an internal salt (n=0) when a single COOH radical is present and forms a CO₂⁻ radical;
it being understood that when R1 denotes a methyl radical and R2 denotes a methyl radical substituted with a —COOH group, then n=1;
and said compound being other than the compounds having the following formula:

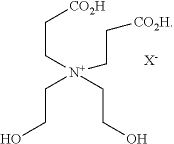

21. A cosmetic process for moisturizing keratin materials, which comprises applying to said keratin materials a composition as defined in claim 16.

22. The cosmetic process according to claim 21, for promoting the persistence of a moisturizing treatment.

23. The cosmetic process according to claim 21, wherein said composition is applied to dry skin and/or to a cutaneous region chosen from:
the hands,
the face,
the neck,
the feet,
the legs,
the arms and forearms.
